(19) United States
(12) Patent Application Publication
(10) Pub. No.: US 2020/0368376 A1
(43) Pub. Date: Nov. 26, 2020

(54) METHOD AND APPARATUS FOR NITROGEN FILLED CHAMBER CARBONIZATION OF WASTE MATERIAL

(71) Applicants: Julius B. CO, Antelope, CA (US); Roderick S. DAYOT, Davao City (PH)

(72) Inventors: Julius B. CO, Antelope, CA (US); Roderick S. DAYOT, Davao City (PH)

(21) Appl. No.: 16/880,770

(22) Filed: May 21, 2020

Related U.S. Application Data
(60) Provisional application No. 62/850,832, filed on May 21, 2019.

(51) Int. Cl. A61L 2/00; A61L 11/00; B09B 3/00; F23G 7/06

(52) U.S. Cl. CPC A61L 2/0094; A61L 11/00; B09B 3/0075; B09B 3/0083; F23G 2200/00; F23G 7/061; A61L 2202/21; F23G 2209/20; A61L 2/0023

(57) ABSTRACT

Example embodiments of the present invention relate generally to medical waste management and more specifically to a method and apparatus for carbonization of waste material using convective heat from the metallic inner surface of a waste treatment chamber and superheated nitrogen gas.

### PRIORITY PATENT APPLICATION

This non-provisional patent application draws priority from U.S. provisional patent application Ser. No. 62/850,832; filed May 21, 2019. The entire disclosure of the referenced patent application is considered part of the disclosure of the present application and is hereby incorporated by reference herein in its entirety.

### COPYRIGHT

A portion of the disclosure of this patent document contains material that is subject to copyright protection. The copyright owner has no objection to the facsimile reproduction of the patent document or the patent disclosure, as it appears in the U.S. Patent and Trademark Office patent files or records, but otherwise reserves all copyright rights whatsoever. The following notice applies to the disclosure provided herein and to the drawings that form a part of this document: Copyright 2019-2020, Julius B. CO and Roderick S. DAYOT; All Rights Reserved.

### TECHNICAL FIELD

Example embodiments of the present invention relate generally to medical waste management and more specifically to a method and apparatus for carbonization of waste material using convective heat from the metallic inner surface of a waste treatment chamber and superheated nitrogen gas.

### BACKGROUND

Waste materials, such as medical waste, biological waste and municipal solid waste made of plastic, cloth, paper, cellulose, etc., are usually burned in incinerators where incomplete combustion is the normal process that produces pungent smoke where to counter such result is done with expensive flue gas treatment processes that would ensure environmental safety and protection. Besides the use of incinerators, another non-burning method of treating medical waste is the use of an autoclave wherein high pressure and high temperature steam is produced from the boiler and is used to disinfect the medical waste through direct injection in the waste chamber. The previously mentioned introduction of steam to the medical waste has been observed to be insufficient in disinfecting the waste since the distribution of heat and heat transfer is uneven during treatment. Furthermore, the steam being applied converts into water at a fast rate such that the moisture from the steam become a wastewater, which requires another treatment, and waste with water from steam becomes wet, heavy, and odorous. Another form of treatment of medical waste is the microwave where the waste material is subjected to microwave radiation for heating. The said heating however needs moisture to excite water molecules to produce heat and the temperature produced is 115 degrees Celsius, which is below disinfection temperature that is 121 degrees Celsius requiring more energy to achieve high temperature. Also, the microwave waste treatment is utilizing a microwave generator that is very sensitive to moisture and can be damaged easily and is expensive. Yet another form of treatment of medical waste is the conventional pyrolysis method where waste is thermally decomposed in the absence of oxygen during heating within a designated chamber resulting in a byproduct of char or carbonized material. The source of heat is a high temperature plasma torch or burner that directly or indirectly heats the waste. The said treatment however cannot achieve a complete oxygen free environment that compromises the pyrolysis process as oxygen is technically present in the system that creates combustion, which is considered incineration. In matters of incineration, medical and hazardous waste, such as plastic materials and other organic materials have different heating values such that they react differently when subjected to burning, such that exhaust gases discharged from these incinerators contain polluting components, like smoke dust, hydrogen chloride, carbon monoxide, Sox, Nox, and heavy metals that includes mercury, dioxin and furan, which are considered harmful pollutants. From the standpoint of environmental protection, it is necessary that formation of these harmful substances should be prevented during waste treatment.

Of these polluting components, dioxin and furan have extremely strong toxicity such that collection and removal of these is extremely important. Plastics from medical waste should therefore have to be conditioned such as drying, shredding and segregating to achieve complete combustion along with the other gases generated by the other organic materials. This will ensure reduction to safety level the toxic gases coming from the exhaust of the incinerator.

Burning of these medical wastes requires high temperature, preferably above 800 to 1200 degrees Celsius, to facilitate complete combustion and elimination of toxic gases generated by such medical waste. However, in conventional incinerators such temperatures cannot be attained since it will require tremendous amount of fuel, thus rendering it to be too costly to do.

An example of the incinerator of the prior art is the conventional refuse incinerator facility, wherein a boiler and auxiliary burner are used. The refuse is directly burned in order to raise the temperature of the incinerator and the temperature of boiling water in the boiler. At the initial start of the operation, there is already a production of a low-temperature combustion gas, which inflicts damage to the facility due to low-temperature corrosion build-up. To solve this problem, the common practice is to discharge this combustion gas by way of a bypass duct and stack. However, there is still the possibility that dust containing hazardous substances, such as dioxin, remain in the incinerator and boiler. If such contamination substances are deposited and still remain in the incinerator, they may be emitted and discharged as gaseous dioxin in the atmosphere even during normal operation.

Another example is an exhaust gas treating apparatus wherein the refuse is incinerated and then completely combusted by a secondary burner in a secondary combustion chamber. The ashes are then discharged to the atmosphere while the exhaust gas generated by the combustion is subjected to heat recovery by a waste heat boiler and waste heat reclaimer (pre-heater) as it flows towards a quenching reaction tower. The exhaust gas in the quenching reaction tower is sprayed with slaked lime slurry to remove hydrogen chloride (HCL) and sulfur oxide (SOx). Smoke dust, fly ash, HCL, SOx, heavy metals and dioxins, which remain in the exhaust gas, are then removed in a bag filter. The exhaust gas after treatment in then discharged to the atmosphere.

Although the exhaust gas from an incinerator is treated with the aforesaid process, there is the possibility that dioxin cannot be reduced to the desired low concentration. Dioxins generated during incineration are almost decomposed in the secondary combustion chamber; however, it is necessary to decrease the temperature of the exhaust gas from a high temperature of about 350 to 900 degree centigrade to a low temperature during every step of the process. However, there is still the tendency of dioxins regenerating at the vicinity of 300 degrees Centigrade during every step of the process such that the above-mentioned conventional exhaust gas treatment apparatus cannot effectively collect and remove dioxins at the desired low concentration.

### SUMMARY

The example embodiments of the present invention as herein disclosed provide treatment of waste materials wherein biodegradable and non-biodegradable waste materials, in particular medical waste, such as plastic, paper, cellulose, etc. are subject to a carbonization process such that high temperature nitrogen gas is utilized as a heat medium to facilitate effective treatment. A nitrogen gas filled treatment chamber is defined in the example embodiments of the present invention as the waste treatment process of heating infectious medical waste and other waste material elements without oxidation through heat transfer from a hot nitrogen gas environment and the convection heat from the surface of the treatment chamber, which is generally made from a heat resistant alloy metal in which the thermally heated nitrogen gas is introduced into the waste chamber that displaces air that contains oxygen and other gas and the waste is effectively treated thermally without burning. The novel process reduces the waste into a gas or changes its phase into a gaseous form because the process is without the presence of air and oxygen. Furthermore, the example embodiments of the present invention are capable of treating such waste through utilization of a heater that raises the nitrogen gas temperature generated by the apparatus. The example embodiments of the present invention further utilize an environmentally compatible non-oxidant, non-combustible gas, such as cheap and readily available nitrogen gas, stable at high temperature in combination with a heating system assisted with an external heater for high temperature treatment. The flue gas being emitted by the example embodiments of the present apparatus is pollutant-free since only nitrogen gas is used, no harmful emission and no chemicals are added in the process.

In view of the aforesaid problems of the prior art, it is therefore an object of example embodiments to provide a method and apparatus for treating waste materials, which can remedy the above-described drawbacks of the prior art including the drawbacks of the present non-burn technologies, such as autoclave, microwave, and pyrolysis methods, which are expensive to manufacture, operate, and maintain.

A further object of example embodiments is to provide an apparatus for treating waste that can be used for heating and treating other waste materials such as garbage and like materials that require thermal decomposition. The example embodiments of the present invention are easy to operate and can be used in cities and industrial facilities for their medical and garbage waste management. Other objects and advantages of the example embodiments of the present invention may be realized upon reading the following description taken in conjunction with the accompanying drawings.

### BRIEF DESCRIPTION OF THE DRAWINGS

Embodiments are illustrated by way of example and not limitation in the figures of the accompanying drawings, in which:

FIG. 1 is an illustrative presentation of an example embodiment of the present invention being used in the oxygen-free and nitrogen-filled chamber for the treatment of waste materials;

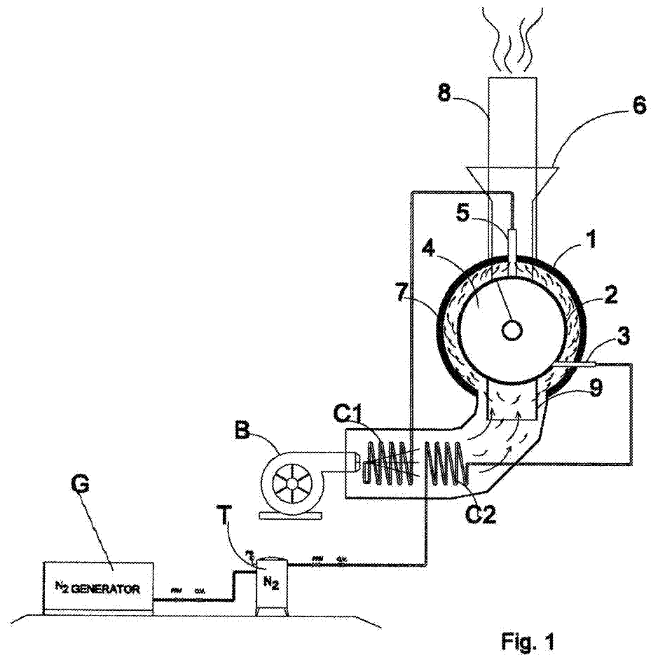

FIG. 2 is a perspective view of one of the example embodiments of the present invention; and

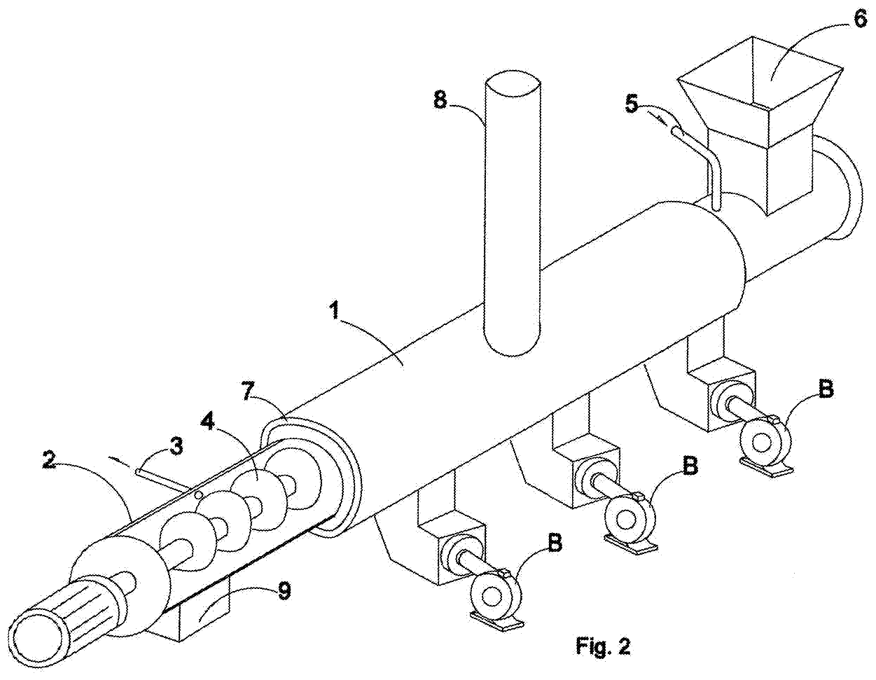

FIG. 3 is a perspective view of the example embodiment of FIG. 1.

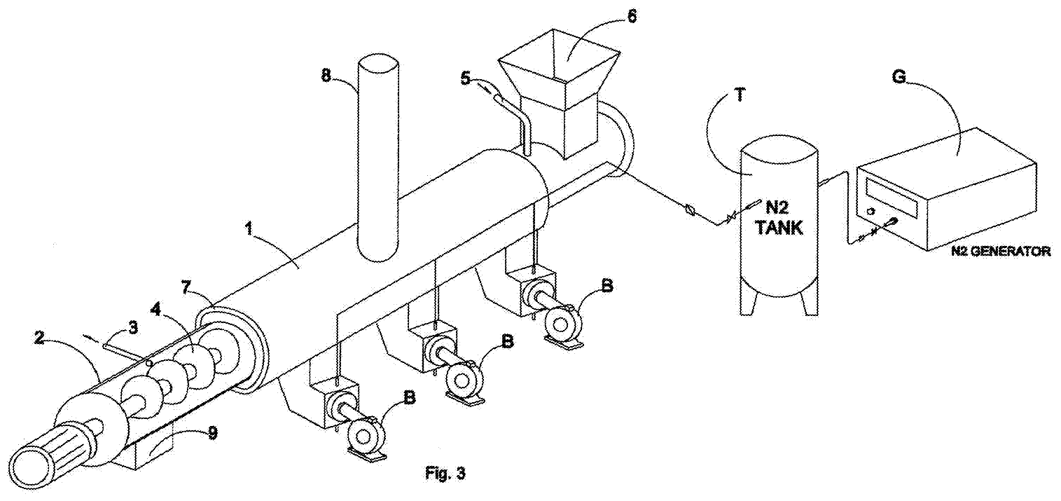

### DETAILED DESCRIPTION

Referring to the drawings, there is shown an apparatus for treating waste, such as medical and hazardous waste and garbage waste, generally designated as 1 comprising an insulated structure 7 having a plurality of heating assemblies B fixedly held on said insulated structure and having a plurality of nitrogen gas port assemblies 3. Said insulated structure being arranged such that it is capable of being held in an elevated manner by suitable conventional support structure (not shown). Insulated structure 7 includes an insulate shell member and hollow inner shell member 2, and a heat chamber being defined by said outer and inner shell members and disposed therebetween. Said inner shell member 2 defining a waste heating chamber, wherein the waste material is capable of passing and indirectly heated therein such that it can be transformed into carbon, and thermally decomposed with a reduction of mass and volume of the treated material. A conveying means 4 disposed within inner shell member 2 being held thereof in a manner wherein it is capable of providing movement of waste material being heated for carbonization. Conveying means 4 is preferably a screw conveyor being capable of rotational movement by suitable prime mover, such as electric motor. A waste material inlet provided on one end of the inner shell member 2 being in communication with a waste hopper 6 fixedly held thereof, and a discharge port 9 provided at the opposite end of said inner shell member 2, wherein the carbonized waste material is being discharged by said conveying means 4. Each of said heating assemblies B includes a pre-heating chamber in communication with said heat chamber, a superheated nitrogen producing means C2 being held on said pre-heating chamber, and a burner B fixedly held on said superheated nitrogen producing means C2. Burner B being arranged in a manner wherein it's capable of providing flame heat in said superheated nitrogen producing means C2, pre-heating chamber and the heat chamber. Said superheated nitrogen producing means C2 is preferably a modified heat exchanger being made such that it is capable of transforming cold nitrogen contained therein into superheated nitrogen through utilization of heat generated by the burner flame. Superheated nitrogen producing means C2 being defined by a nitrogen containing means mounted on a high temperature nitrogen chamber C2, a superheated nitrogen line 3 having a coiled section C2 disposed within the high temperature nitrogen chamber and in communication with said nitrogen containing means T. Superheated nitrogen line 3 is preferably made of chromium and nickel such that it can serve as better passage of the superheated nitrogen upon contact thereof. Said high temperature nitrogen gas is then being introduced to the treatment chamber through gas discharge port of the superheated nitrogen line 3 to increase the air/oxygen displacement activity and removing all air inside the treatment chamber. During the treatment, a hydrocarbon gas line 5 disposed within the pre-heating chamber and in communication with said waste heating chamber, such that the hydrocarbon gas produced within the waste heating chamber is capable of flowing thereof and into the pre-heating chamber. Hydrocarbon gas derived from decomposed waste gas line 5 having a coiled section C1 disposed within the pre-heating chamber such that the hydrocarbon gas is subjected to further heating by the flame of the burner before being introduced to the heat chamber through a gas outlet provided thereof. Such introduction of hydrocarbon gas in the heat chamber allows combustion of gases in the waste heating chamber and provides additional means for further increasing the heating temperature during the heating process.

The insulated structure 7 may be made to communicate with at least another identical insulated structure such that further carbonization can be facilitated. An air-cooling chamber may be provided (not shown) and being made to communicate with one of said insulated structure to facilitate cooling and disposing the carbonized waste material coming from the said insulated structure. Furthermore, a gas filtering means may be provided and arranged to communicate with said insulated structure through the port 8 such that filtering of exhaust gases such as hydrocarbon gas, carbon monoxide, and hydrogen, produced in the waste chamber during the heating of the waste material may be converted into carbon dioxide and water. In one arrangement, gas-filtering means includes a draft fan for drawing out the gases, an activated carbon filter (not shown) for filtering the gases and a cyclonic enclosure (not shown) in communication with said filtering means for converting said gases to water and carbon dioxide.

The method for treating waste material of an example embodiment includes among the following steps or actions. Heat nitrogen at a high temperature until it is transformed into superheated nitrogen. Said heating can be facilitated by suitable burning means, such as burner or any source of heat energy B. The superheated nitrogen is then allowed to pass through a pipe made with chromium and nickel such that the said superheated nitrogen which in turn is re-introduced in the waste treatment chamber, thereby increasing the heat transfer from nitrogen to the waste and at the same time displacing all oxygen containing gas inside the chamber. Said high temperature heat in the heat chamber is then indirectly transferred to the waste heating chamber to facilitate carbonization of the waste being treated. When treating biodegradable and non-biodegradable medical or garbage waste material, such as plastic, wood, paper, cellulose, etc., the temperature within the waste heating chamber should be at least above 500 degrees Celsius such that at the said temperature, the waste can be converted into carbonized form and there would be production of waste derived hydrocarbon gas through a high temperature pyrolysis process. The pyrolysis process is the process of heating fuels and other combustible elements without oxidation. The thermally treated solid waste in this case is converted into fuel and changes its phase into a gaseous form without the presence of air and oxygen. This is made possible by the presence of nitrogen gas inside the treatment chamber. The gas produced is called hydrocarbon gas that can also be used as fuel. The hydrocarbon gas produced in the waste chamber during the heating process of the waste materials is then introduced as fuel and allowed to mix with the flame in combination with the superheated nitrogen. The process effectively consumes the hydrocarbon gas, which is considered a pollutant. Since the treatment utilizes a combination of pyrolysis derived gas and nitrogen injection process, a cleaner emission is achieved since it only uses nitrogen and no chemicals are used in the process. Furthermore, such recycling of pyrolysis-nitrogen filled generated treated waste derived gas further serves in reducing the amount of fuel spent during the burning/heating process.

We claim:

1. A method for treating waste material, the method comprising:
heating nitrogen gas at a pre-determined high temperature until it is converted into superheated nitrogen gas;
allowing said superheated nitrogen gas be in contact with the waste inside the heating chamber while at the same time displacing the air inside the treatment chamber;
heating of the waste thermal treatment chamber;
introducing the high temperature nitrogen gas into the waste thermal treatment chamber;
introducing said nitrogen gas and hydrocarbon gas coming from the treatment chamber into the flame being used for heating said nitrogen gas such that the heating value of the heat being generated by said flame is increased;
heating waste material by said heat of increased heating value;
collecting hydrocarbon gas produced during heating of said waste material;
introducing said hydrocarbon gas into the said flame as fuel, thereby removing highly potential pollutants in the gas waste being treated; and
carbonizing the waste materials through nitrogen gas filled chamber and heating the chamber increasing its inside temperature and pressure.

2. A method for treating waste according to claim 1, wherein the temperature of said heat with high heating value is about 400 to 700 degrees Celsius.

3. A method for treating waste material according to claim 1, wherein said waste material is medical waste.

4. A method for treating waste material according to claim 3, wherein the temperature of heat with high heating value is 121 degree Celsius when the waste material being heated is medical waste such that it can be disinfected and pathogens can be eliminated thereof.

5. A method for treating waste material according to claim 1, wherein the waste is indirectly heated by said heat with high heating value.

6. An apparatus for treating waste material, the apparatus comprising:
an insulated structure having a plurality of heating assemblies being held thereof, each of said heating assemblies being arranged in a manner wherein it is capable of providing flame heat in said insulated structure, said insulated structure being made such that it is capable of accommodating and disposing waste materials being heated and carbonized therein, insulated structure includes an insulated hollow outer shell member and hollow inner shell member that defines a heating chamber disposed therebetween, a waste heating chamber being defined by said inner shell member, and conveying means provided within the inner shell member being held thereof in a manner wherein it is capable of providing movement and disposal of the heated and/or carbonized waste materials disposed in the inner shell member, each of said heating assemblies consisting of a pre-heating chamber in communication with said heating chamber, a superheated nitrogen producing means in communication with said pre-heating chamber, and a burner fixedly held on said superheated nitrogen producing means and being arranged such that it is capable of providing flame heat in said superheated nitrogen producing means, pre-heating chamber and heating chamber, said superheated nitrogen producing means being capable of transforming nitrogen contained therein into superheated nitrogen through utilization of heat generated by said burner and facilitates introduction of superheated nitrogen gas into the burner flame in the pre-heating chamber, and hydrocarbon gas line in communication with said waste heating chamber being arranged such that it is capable of allowing the introduction into the burner flame the hydrocarbon gas produced during heating of the waste material within the waste heating chamber.

7. An apparatus for treating waste material according to claim 6, wherein said superheated nitrogen producing means is a heat exchanger made from coiled pipe medium.

8. An apparatus for treating waste material according to claim 6, wherein said superheated nitrogen pipeline being made of chromium and nickel alloy heat resistant pipe such that it can serve as corrosion resistant upon contact of said superheated nitrogen thereof and is insulated outside with heat resistant insulating material.

9. An apparatus for treating waste material according to claim 6 wherein said conveying means is a screw conveyor.

10. An apparatus for treating waste material according to claim 8, wherein said superheated nitrogen line having a coiled section disposed within the pre-heating chamber.

11. An apparatus for treating waste material according to claim 6, wherein said nitrogen gas line having a coiled section disposed within the pre-heating chamber.

12. An apparatus for treating waste material according to claim 6, wherein said insulated structure being in communication with an identical insulated structure such that further carbonization and/or disinfection of waste could be facilitated.

13. An apparatus for treating waste material according to claim 12, wherein an air cooling chamber is in communication with one of said insulated structure being made such that it is capable of cooling and disposing of the carbonized and/or disinfected waste from said insulated structure.

14. An apparatus for treating waste material according to claim 12, wherein a gas filtering means is in communication with said insulated structure being made such that it is capable of filtering the gases produced in the said waste chamber during the heating of said waste materials, such as hydrocarbon gas and hydrogen, thereby converting such gases into carbon dioxide and water.
